(19) United States
(12) Patent Application Publication
(10) Pub. No.: US 2005/0170484 A1
(43) Pub. Date: Aug. 4, 2005

(54) Continuous enzymatic desizing method

(76) Inventor: Kurt Von Wersch, Wegberg (DE)

Correspondence Address: WILLIAM COLLARD;COLLARD & ROE, P.C., 1077 NORTHERN BOULEVARD, ROSLYN, NY, 11576, US

(21) Appl. No.: 10/508,271

(22) PCT Filed: Dec. 6, 2003
(86) PCT No.: PCT/DE03/04023

(30) Foreign Application Priority Data
Jan. 28, 2003 (DE) 103 03 328.9

(51) Int. Cl. C12N 9/00

(52) U.S. Cl. 435/183

(57) ABSTRACT

A method for continuous de-sizing of starch-sized woven textiles is described. In this connection, the woven textile is dipped into a bath containing enzyme mixtures as well as bacterial alpha amylases, having a pH of 5 to 7.5, and then treated at an elevated temperature. De-sizing becomes particularly uncomplicated if the woven textile that was treated with the bath in a foulard, in other words the damp woven textile, is passed into an air dryer through which it passes continuously, in which an atmosphere of approximately 120° Celsius and approximately 25 to 30 vol.-% steam content is formed.

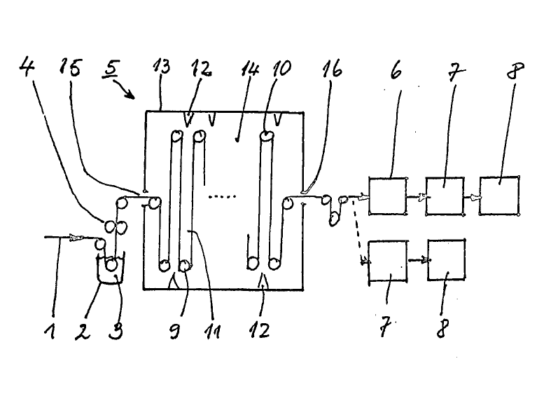

The invention relates to a method for continuous enzymatic de-sizing of starch-sized woven textiles, dipped into a bath containing enzyme mixtures and bacterial alpha amylases, having a pH of 5 to 7.5, at an elevated temperature.

A method of this type is described in a brochure from the Cognis company, under the key word Forylase AT; “Forylase” is a registered trademark. According to the information in the brochure, Forylase is a bacterial alpha amylase that is suitable for enzymatic de-sizing of starch-sized woven textiles. It is intended for use in continuous operation. According to information provided by Cognis, the bacterial amylase assures quick and complete decomposition of starch into water-soluble substances, and develops its effectiveness in a bacterial action temperature range between 60 and 100° Celsius, at a pH of 5.5 to 7.5. For the known continuous method, a batch with 2 to 4 ml/l bacterial alpha amylase, among other things, is prepared. The woven textile to be de-sized is dipped into the bath and treated with saturated steam for two to five minutes, in a pad/steam system, at 100° Celsius, and subsequently washed in a washing machine at 95° Celsius. Operation of the pad/steam system is complicated.

Another continuous method for de-sizing cellulose goods in strip shape is described in WO 00/17437. In this method, a treatment bath containing per compounds is applied to the cellulose goods, in order to combine de-sizing with boiling-out. In many cases, the aggressive per compounds are not desirable, e.g. for reasons of environmental protection.

In EP 07 97 698 B1, a device for color fixing during reactive dyeing of cellulose goods is described. In this method, a dye bath that contains the reactive dye is applied to the goods with a foulard. An air dryer, e.g. a hot flue, follows the foulard. The air dryer possesses a chamber having a goods inlet and outlet, as well as having a controllable circulation ventilator, as well as means for regulating predetermined water content in the ambient air. In this way, the result is supposed to be achieved that the reactivity of the dyes used results in an optimal dye yield, as a function of the drying of the goods, without aggressive chemicals being required. However, this method is neither intended nor suitable for de-sizing.

The invention is based on the task of creating a continuous method for de-sizing goods treated with sizing that contains starch, in which neither aggressive chemicals nor complicated pad/steam systems are required.

The solution according to the invention is described in claim 1. For the method indicated initially, it preferably consists in the fact that the de-sizing of the woven textile that was treated with the bath (which contains the enzyme mixtures as well as bacterial alpha amylases) in a foulard, in other words the damp woven textile, takes place in an air dryer through which it passes continuously, in which an atmosphere of approximately 120° Celsius and approximately 25 to 30 vol.-% steam content is formed, which results in an action temperature of approximately 68 to 70° Celsius at the woven textile. The action temperature (or bacterial action temperature) is the temperature at which the stated bacteria are in action in the sense of de-sizing. Some improvements and additional embodiments of the invention are indicated in the dependent claims.

The invention is based on the recognition by the inventor that the bacteria in alpha amylases as well as enzyme mixtures do particularly well in the indicated, narrow action temperature range, at 30 vol.-% steam, so that they are capable and desirous of quickly and completely decomposing the starch in the case of starch-sized woven textiles, in a time frame of two minutes, without harming the woven textile or its fibers.

The air dryer provided according to the invention can, in principle, be configured as described in EP 07 97 698 B1. At its core, the invention also consists of using a dryer, e.g. a hot flue or a stentering frame, for de-sizing woven textiles sized with starch, in other words using machines that were originally designed for a completely different purpose. The invention can therefore also be described as the use of such an air dryer for de-sizing a web of woven textile that previously had the de-sizing bath containing the enzyme mixtures and the bacterial alpha amylases applied to it in a foulard.

In practice, denim warps, for example, are treated with sizing that contains starch. The woven textiles to be de-sized according to the invention are preferably denim goods. These goods consist of a sized and indigo-dyed warp and generally a white weft. According to the invention, the webs of woven textile can be dried at the prescribed relative humidity and temperature, after foularding (with the enzyme/alpha amylase bath), and then either washed immediately, then sewn, and finally effect-washed as a sewn article of clothing, or only sewn after drying and then washed/effect-washed.

Details of the invention will be explained using the schematic representation of an exemplary embodiment.

The woven textile 1 that comes from the weaving mill is passed through a bath 3 in a foulard 2, subsequently the water is removed in a squeezer 4, and then the textile is dried in an air dryer 5 at an air temperature of approximately 120° Celsius and approximately 25 to 30 vol.-% steam content. In this atmosphere, the damp woven textile has a so-called cooling limit temperature of 68 to 70° Celsius, which remains constant for a sufficiently long time for the action of the bacteria used, independent of the current moisture content of the woven textile. Subsequent to the air dryer 5, the woven textile can be passed through a first washing machine 6, the sewing station 7, and finally, as an article of clothing, through a second washing machines 8, for effect washing. Alternatively, the unwashed but de-sized woven textile 1 can be passed directly to the sewing station and subsequently passed through the washing station having a second washing machine 8. The woven textile 1 reaches the second washing machine 8 no longer as a web but rather as a finished, sewn article of clothing.

If the air dryer 5 is a hot flue (as shown), the woven textile 1 is passed over a plurality of upper and lower guide rollers 9 and 10, respectively, in loops 11, and blown on from jets 12, from below and above. By means of the jets, ambient air at a temperature of 120° Celsius and the prescribed humidity of 25 to 30 vol.-% steam is blown into a chamber 14 of the air dryer 5, surrounded by a housing 13. To provide a seal, the housing 14 possesses an inlet slit 15 and an outlet slit 16 for the web of woven textile.

Reference Symbol List:

1=woven textile
2=foulard
3=bath
4=squeezer
5=air dryer
6=washing machine
7=sewing station
8=washing machine
9=lower guide roller
10=upper guide roller
11=loop of textile web
12=blowing jet
13=housing
14=chamber
15=inlet slit
16=outlet slit

1. Method for continuous enzymatic de-sizing of starch-sized woven textiles (1), dipped into a bath (3) containing enzyme mixtures and bacterial alpha amylases, having a pH of 5 to 7.5, at an elevated temperature, characterized in that the woven textile (1) is foularded and formed in an air dryer through which it passes continuously and which is held at an atmosphere of about 120° C. and 25 to 30 vol. % of water vapor, which produces an action temperature of about 68 to 70° C. on the damp woven textile (1).

2. Method according to claim 1, characterized by the use of a hot flue as the air dryer.

3. Method according to claim 1, characterized by the use of a stentering frame as the air dryer.

4. Method according to claim 1, characterized by its use for denim goods.
